(19) United States
(12) Patent Application Publication
(10) Pub. No.: US 2024/0298961 A1
(43) Pub. Date: Sep. 12, 2024

(54) METHOD AND APPARATUS FOR DETERMINING USER ANTHROPOLOGICAL TYPE TO REFINE ESTIMATION OF USER'S PHYSIOLOGICAL PARAMETERS

(71) Applicant: Samsung Electronics Co., Ltd., Suwon-si (KR)

(72) Inventors: Alexey Viacheslavovich PERCHIK, Moscow (RU); Konstantin Aleksandrovich PAVLOV, Moscow (RU); Vladimir Germanovich TSEPULIN, Moscow (RU); Jaehyuck PARK, Suwon-si (KR); Wonseok LEE, Suwon-si (KR); Namseok CHANG, Suwon-si (KR)

(21) Appl. No.: 18/667,580

(22) Filed: May 17, 2024

Related U.S. Application Data
(63) Continuation of application No. PCT/KR2022/019490, filed on Dec. 2, 2022.

(30) Foreign Application Priority Data
Dec. 17, 2021 (RU) 2021137525

(51) Int. Cl. A61B 5/00

(52) U.S. Cl. CPC A61B 5/442; A61B 5/443

(57) ABSTRACT

The disclosure relates to a method and an apparatus for determining anthropological type of a user is provided. The apparatus includes a spectral optical sensor, a thickness sensor, memory storing one or more computer programs, and one or more processors communicatively coupled to the spectral optical sensor, the thickness sensor and the memory wherein the one or more computer programs include computer-executable instructions that, when executed by the one or more processors, cause the apparatus to calculate an overall melanin concentration value and a melanin concentration ratio value of a user's skin, compare the obtained scattering intensity values with values of scattering intensity corresponding to a special skin tone from a database of simulated data, compare the obtained thickness value with data of skin thickness from the database of simulated data, compare the calculated overall melanin concentration value and the calculated melanin concentration ratio value of the user's skin with data of an overall melanin concentration and a melanin concentration ratio from the database of simulated data, and assign an anthropological type to the user in accordance with the selected skin sample.

### CROSS-REFERENCE TO RELATED APPLICATION(S)

This application is a continuation application, claiming priority under § 365(c), of an International application No. PCT/KR2022/019490, filed on Dec. 2, 2022, which is based on and claims the benefit of a Russian patent application number 2021137525, filed on Dec. 17, 2021, in the Russian Intellectual Property Office, the disclosure of which is incorporated by reference herein in its entirety.

### BACKGROUND

### 1. Field

The disclosure relates to a field of health status monitoring. More particularly, the disclosure relates to methods and apparatuses for monitoring state of body, used in fitness.

### 2. Description of Related Art

Currently fitness is a popular and in-demand type of physical activity to maintain a human's health and good physical condition. Maintaining general physical condition is achieved through proper nutrition, rest, and moderate physical activity.

A popular type of health status monitoring is the use of wearable devices, such as, for example, fitness bracelets or smart watches. Well-known wearable devices can estimate the pulse, pressure, temperature, fluid loss by the user, or the like. Since, by attending fitness, the user is involved in determining the state of his health, it is desirable to ensure an accurate determination of the parameters associated with determining the health of the user, while it is necessary to take into account, among other things, the anthropological type of the user, since the parameters of the norm for different anthropological types differ. Since there are differences between different anthropological types, such as, in particular, the distribution of sweat glands, muscle distribution, body proportions, or the like, the anthropological type of user is an important parameter, for example, for calculating the necessary physical activity, as well as for selecting the required number of calories of nutrition. However, it is currently considered unethical to ask the user direct questions concerning his belonging to one or another anthropological type, such direct questions may be negatively perceived by some users, up to the user's refusal to use the device asking such questions. Therefore, there is a need to create a device that will independently determine the anthropological type of user.

From the prior art, see document US20060210154 (publication date 21 Sep. 2006), a skin tone estimating device is known. The device includes a measuring head located opposite the skin. The measuring head includes an output window through which light should enter the skin, and a detection window configured to receive light emitted through the output window. The distance between the exit window and the detection window can be selected so that at least the main part of the light emitted from the exit window passes through the skin at a distance of at least 1 mm before reaching the detection window. The disadvantage of the known device is the inability to obtain any other information about the user, except for skin tone, including with the help of this known device, information about the composition of chromophores necessary for predicting the anthropological type of a human cannot be obtained.

From the prior art, see document U.S. Pat. No. 8,861,847 (publication date 14 Oct. 2014), a system for adaptive skin tone detection from one or more images is known. The system includes an image processing module configured to receive images and provide increased image contrast to compensate for background illumination in the image. The image processing module is additionally configured to detect and identify areas of the image with high contrast containing a shade of human skin, based on the use of multiple color spaces and adaptively generated threshold values for each color space. The disadvantage of the known device is the inability to use it in wearable devices. The information about the composition of chromophores necessary to predict the anthropological type of the user cannot be obtained.

Thus, it is necessary to create a compact wearable device that can automatically detect the user's anthropological type, and with which the user can determine his physiological parameters, taking into account his anthropological type.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a method and an apparatus for determining user anthropological type to refine estimation of user's physiological parameters.

In an accordance with an aspect of the disclosure, an apparatus for determining anthropological type of a user is provided. The apparatus incudes a spectral optical sensor configured to emit radiation on a user's skin and obtain scattering intensity values for the radiation on the user's skin, a thickness sensor configured to obtain a thickness value of the user's skin, memory storing one or more computer programs, and one or more processors communicatively coupled to the spectral optical sensor, the thickness sensor, and the memory, wherein the one or more computer programs include computer-executable instructions that, when executed by the one or more processors, cause the apparatus to calculate an overall melanin concentration value and a melanin concentration ratio value of the user's skin based on the obtained scattering intensity values and the obtained thickness value, compare—the obtained scattering intensity values with values of scattering intensity corresponding to a special skin tone from a database of simulated data, to select a set of skin sample corresponding to the special skin tone from the database of simulated data,—the obtained thickness value with data of skin thickness from the database of simulated data, to select skin samples having the closest values with the obtained thickness value from the selected set of skin sample, and—the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples, and assign an anthropological type to the user in accordance with the selected skin sample.

In an accordance with an aspect of the disclosure, a method for operating an apparatus for determining anthropological type of a user is provided. The method includes emitting radiation on a user's skin using the spectral optical sensor, obtaining scattering intensity values for the radiation on the user's skin, using the spectral optical sensor, obtaining a thickness value of the user's skin using the thickness sensor, calculating, using the processor, an overall melanin concentration value and a melanin concentration ratio value of the user's skin based on the obtained scattering intensity values and the obtained thickness value, comparing, in the processor the obtained scattering intensity values with values of scattering intensity corresponding to a special skin tone from a database of simulated data, to select a set of skin sample corresponding to the special skin tone from the database of simulated data,—the obtained thickness value with data of skin thickness from the database of simulated data, to select skin samples having the closest values with the obtained thickness value from the selected set of skin sample, and the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples, and assigning an anthropological type to the user in accordance with the selected skin sample.

In accordance with another aspect of the disclosure, one or more non-transitory computer-readable storage media storing computer-executable instructions that, when executed by one or more processors of an apparatus, cause the apparatus to perform operations are provided. The operations include calculating an overall melanin concentration value and a melanin concentration ratio value of a user's skin based on the obtained scattering intensity values and the obtained thickness value, comparing the scattering intensity values with values of scattering intensity corresponding to a special skin tone from a database of simulated data, to select a set of skin sample corresponding to the special skin tone from the database of simulated data, the thickness value with data of skin thickness from the database of simulated data, to select skin samples having the closest values with the obtained thickness value from the selected set of skin sample, and the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples, and assigning an anthropological type to the user in accordance with the selected skin sample.

Additional aspects will be set forth in part in the description which follows and, in part, will be apparent from the description, or may be learned by practice of the presented embodiments.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates a scheme of determining anthropological type of a user through an optical spectrum and a skin thickness according to an embodiment of the disclosure;

FIG. 2A illustrates a location of a photoplethysmogram sensor (PPG) in a smart bracelet and an operation of a PPG sensor according to an embodiment of the disclosure;

FIG. 2B schematically illustrates an operation of a PPG sensor according to an embodiment of the disclosure;

FIG. 3A illustrates chemical formulas of eumelanin and phaeomelanin according to an embodiment of the disclosure;

FIG. 3B illustrates an absorption spectra of eumelanin and phaeomelanin according to an embodiment of the disclosure;

FIG. 4 illustrates a skin tone detection algorithm for two different users according to an embodiment of the disclosure;

FIG. 5 illustrates a determination of anthropological type of a user depending on an overall melanin concentration and a ratio of eumelanin and phaeomelanin according to an embodiment of the disclosure;

FIG. 6 schematically illustrates an operation of a smart bracelet to measure physiological parameters according to an embodiment of the disclosure;

FIG. 7 is a diagram illustrating an apparatus for determining anthropological type of a user according to an embodiment of the disclosure; and

FIG. 8 is a diagram for describing a method for operating an apparatus for determining anthropological type of a user according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

In accordance with an aspect of the disclosure, for example, using of a smart bracelet to determine user's anthropological type, to apply the data of user's anthropological type later on while determining user's physiological parameters. The device is compact and easy to handle, the device allows to determine the user's anthropological type, for example, with the help of a smart bracelet.

The anthropological type of a human-a historically formed complex of hereditarily persistent signs of physical structure that characterize groups of people in a certain territory. The anthropological type denotes both large races and their subgroups.

The composition of the human body means the amount of adipose tissue, the amount of muscle tissue. Moreover, it is known that the average statistical data of the human body composition depends on the anthropological type to which the human belongs. It is known that all anthropological types are grouped into three large groups: equatorial, Caucasoid and Mongoloid.

Following parameters are embedded in the anthropological type of a human: melanin concentration in the skin, concentration ratio of different melanins, physique, distribution of sweat glands, distribution of muscles, body proportions, or the like.

Calculations of body composition based on impedance are specific to different groups of people whose type contains a certain set of parameters (genetic characteristics), since differences in physique are observed in different such groups, such as, for example, the relative length of arms and legs.

The disclosure can be used in any suitable user computing device containing a processor and memory, storing instructions for execution steps of the proposed method by the processor. Such a device may be, but without limitation, a smartphone, smartwatch, and other suitable user-wearable devices, which will be hereinafter referred to as a smart bracelet. The memory may be any medium for storing data, in particular a computer-readable data carrier.

The disclosure is based on the analysis of individual user data obtained from the sensors used in a smart bracelet. Sensors can be inertial, optical, electrical, temperature, or the like, the presence of a set of such sensors is sufficient for the implementation of the disclosure.

When collecting data, the following necessary sensors are used: spectral optical sensor, such as, for example, optical pulse sensor (photoplethysmograph), sensor skin thickness determination. In addition to data from sensors, individual user data is used, such as the user's age, sex, weight, and height. It is known from the prior art that the height, weight, age, and sex of the user affect skin thickness, for example, publication Shuster S, Black M M, McVitie E. The influence of age and sex on skin thickness, skin collagen and density. Br J Dermatol. 1975 December; 93(6):639-43. doi: 10.1111/j.1365-2133.1975.tb05113.x. PMID: 1220811 and publication Black M M, Bottoms E, Shuster S. Skin collagen and thickness in simple obesity. Br Med J. 1971 Oct. 16;4(5780):149-50. doi: 10.1136/bmj.4.5780.149. PMID: 5113017; PMCID: PMC1799028.

It should be appreciated that the blocks in each flowchart and combinations of the flowcharts may be performed by one or more computer programs which include computer-executable instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g., a central processing unit (CPU)), a communication processor (CP, e.g., a modem), a graphical processing unit (GPU), a neural processing unit (NPU) (e.g., an artificial intelligence (AI) chip), a wireless-fidelity (Wi-Fi) chip, a Bluetooth™ chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

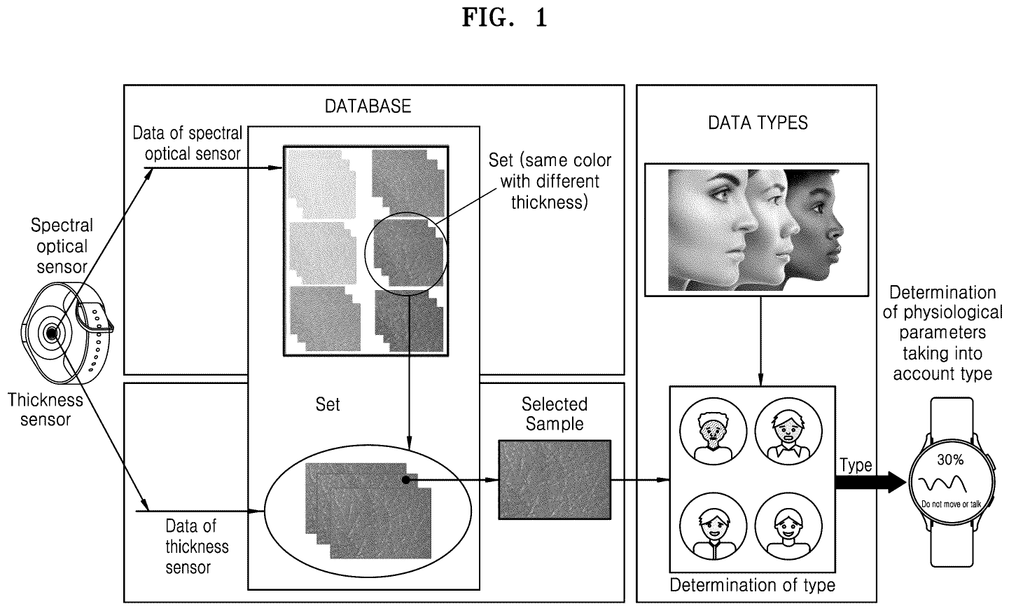

FIG. 1 illustrates a scheme for determining a user's anthropological type using an optical spectrum of radiation from a skin and a skin thickness according to an embodiment of the disclosure.

Referring to FIG. 1, a smart bracelet comprises a spectral optical sensor. Such sensors are known from the prior art, for example, Pulse Oximeter and Heart-Rate Sensor IC for Wearable Health: https://www.maximintegrated.com/en/products/sensors/MAX30100.html. The smart bracelet also comprises sensor for skin thickness determination, the skin thickness determination technique using an optical sensor is given, for example, in Wissel T, Bruder R, Schweikard A, Ernst F. Estimating soft tissue thickness from light-tissue interactions—a simulation study. Biomed Opt Express. 2013 Jun. 14;4(7):1176-87. doi: 10.1364/BOE.4.001176. PMID: 23847741; PMCID: PMC3704097. The data obtained from the spectral optical sensor and thickness sensor are compared with data stored in a database containing pre-modeled skin data of different anthropological types. Samples are selected from the database, having skin tone and skin thickness, which correspond to the skin tone and skin thickness of the user.

It should be noted that people with different skin thickness may have the same skin tone, which is due to different concentrations of chromophores, including melanins and other substances. The melanin concentration per unit volume is a parameter directly related to the anthropological type of a human. However, the skin of people with the same skin tone and the same thickness may have a different melanin concentration ratio and belong to people of different anthropological types.

The database is modeled (formed) in advance based on the data known from the prior art of color matching, skin thickness, and the ratio of melanin concentrations determined by the anthropological type, see for example, Huang W S, Wang Y W, Hung K C, Hsieh P S, Fu K Y, Dai L G, Liou N H, Ma K H, Liu J C, Dai N T. High correlation between skin color based on CIELAB color space, epidermal melanocyte ratio, and melanocyte melanin content. PeerJ. 2018 May 24;6: e4815. doi: 10.7717/peerj.4815. PMID: 29844968; PMCID: PMC5971097. The database of simulated data includes sets of skin samples, where each sample corresponds to an anthropological type. Each set corresponds to a certain one skin tone, and each such set contains samples having different values of skin thickness for the same skin tone. At the same time, each sample having a certain color and a certain skin thickness has its own unique melanin concentration ratio value and their overall concentration, which are calculated in the calculation unit based on the data received from the spectral optical sensor, and each mentioned unique value corresponds to its anthropological type. Moreover, the calculation unit calculates the value of the overall melanin concentration and a melanin concentration ratio value in the human skin based on the intensity values obtained from each light-emitting diode (LED) and the thickness value obtained from the thickness sensor.

The skin of the known anthropological type of a human corresponds to the known melanin content defining the skin tone, some skin thickness and a predetermined melanin concentration ratio and overall melanin concentration. The melanin content corresponds to the known scattering spectrum, particularly the peaks of scattering occurring on the skin for radiation incident on the skin, obtained by a spectral optical sensor. Thus, data of scattering peaks on the skin corresponding to a specific skin tone with a known skin thickness, with a known overall melanin concentration with a known ratio of melanin concentrations are entered into the database, which corresponds to belonging to a specific anthropological type, thus sets of skin samples are formed.

The skin tone of the user is determined using spectral measurements using a spectral optical sensor in the form of, for example, a photoplethysmogram sensor (PPG sensor). Each of the ranges of intensity values received from the spectral optical sensor corresponds to a specific skin tone, that is, corresponds to its own set of samples with the same skin tone, but with different skin thickness values. When a set of samples from the database with one skin tone is selected based on the values obtained from the spectral optical sensor, the data of skin thickness obtained from the thickness sensor is compared with the data of skin thickness of the samples of the selected set, since different skin thickness in different anthropological types can correspond to the same skin tone. For example, if a user belonging to the Caucasian anthropological type tans, then his skin tone may coincide with the skin tone of a user of the equatorial anthropological type, however, skin thickness, as well as the melanin concentration ratio will correspond exactly to the Caucasian anthropological type.

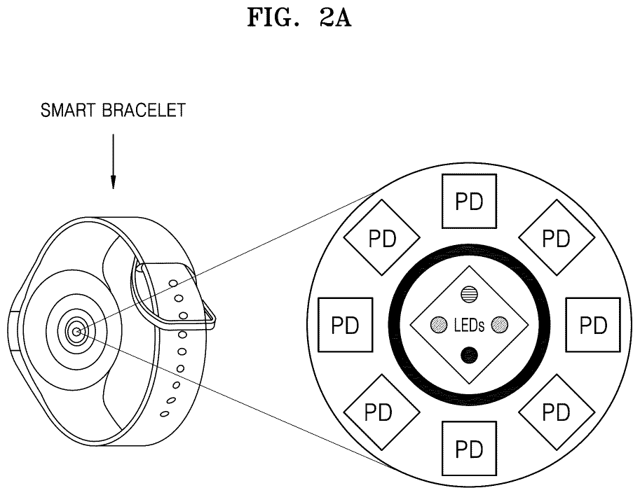

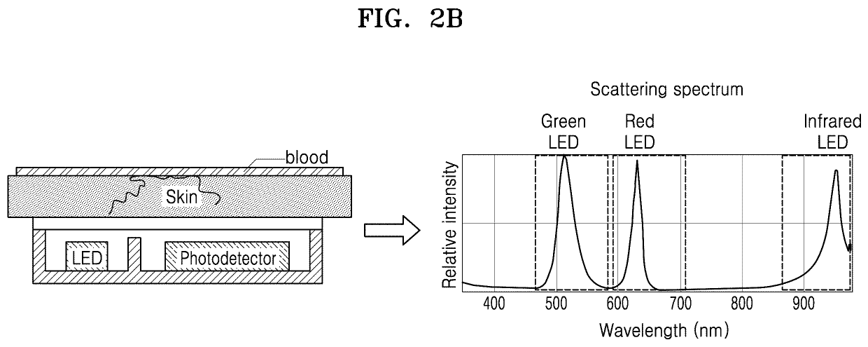

FIG. 2A illustrates a location of a spectral optical sensor on a side of a smart bracelet facing a user's skin according to an embodiment of the disclosure.

Referring to FIG. 2A, spectral optical sensor consists of photodetectors (PDs) and light emitting diodes (LEDs). Red light emitting diodes (R), green light emitting diodes (G), infrared light emitting diodes (IR) are used.

As shown in FIG. 2B, light emitting diodes (LED) alternately emit radiation that hits the user's skin, the radiation is scattered and reflected by the skin.

FIG. 2B illustrates scattering peaks for radiation arriving to a skin from red, green, and infrared LEDs according to an embodiment of the disclosure.

Referring to FIG. 2B, by the ratio of intensity values in the scattering peaks, it is possible to get information about skin color, and knowing the value of skin thickness, it is possible to get information about the overall melanin concentration in the skin of the user and the melanin concentration ratio, that determine the anthropological type.

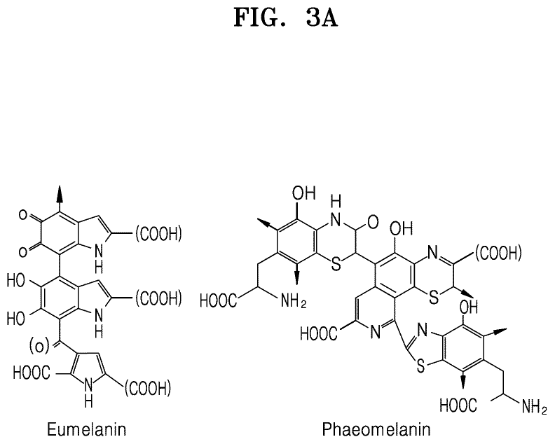

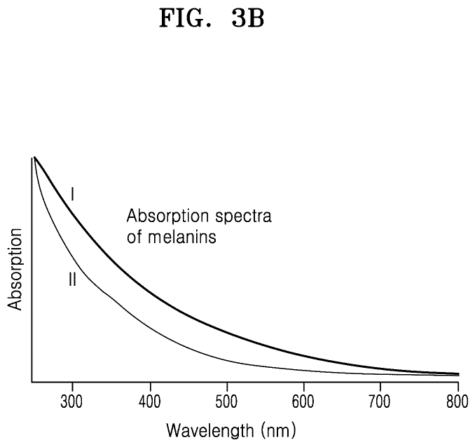

FIG. 3A illustrates chemical formulae of, eumelanin and phaeomelanin being two types of melanin, presenting in the skin according to an embodiment of the disclosure.

Referring to FIG. 3A, the phenomenon that different substances absorb radiation at different wavelengths (light of different colors) differently underlies the operation of all spectral tools, allowing in particular to determine the radiation scattered by melanins. It is known that different anthropological types have different concentration ratio for eumelanin and phaeomelanin in the skin.

FIG. 3B illustrates an absorption spectra of eumelanin and phaeomelanin according to an embodiment of the disclosure.

Referring to FIG. 3B, the absorption spectra of eumelanin and phaeomelanin, demonstrate that eumelanin (curve I) and phaeomelanin (curve II) absorb radiation differently, and also that the ratio of absorption of eumelanin I to absorption of phaeomelanin II at the same wavelength differs from the ratio of absorption of eumelanin I to absorption of phaeomelanin II at a different wavelength.

By irradiating eumelanin and phaeomelanin, located in the user's skin, sequentially with radiation at the wavelengths of a spectral optical sensor, that is, radiation from a red LED, a green LED, and an infrared LED, three scattering intensity values of radiation corresponding to the peaks of the spectral curve at these wavelengths (FIG. 2B) are obtained, and each ratio of the intensities of these three the peaks correspond to the specific ratio of eumelanin and phaeomelanin concentration and depending on skin thickness. For example, the concentration ratio for eumelanin and phaeomelanin and the overall melanin concentration are calculated from the ratio of the intensities of the scattering peaks and skin thickness in the calculation unit.

Next, using the machine learning algorithm, implemented in the comparison unit, the database of simulated data, divided into sets, is traced. Each set corresponds to specific skin tone, wherein said specific skin tone from each set of skin samples corresponds to specific values of scattering intensities for radiation arriving from light emitting diodes.

Each of the sets comprises samples having the same skin tone, but different skin thickness values. Using the algorithm, a set with overall melanin concentration is found in the database of simulated data, as close as possible to the calculated values of scattering intensities of overall melanin concentration, which corresponds to a specific skin tone. Further, having information about the user's skin thickness obtained from the thickness sensor, samples are selected from the selected set in which the skin thickness is as close as possible to the user's skin thickness. Each of the samples is characterized by its own unique values of the concentration of phaeomelanin and eumelanin, that is, the unique value of the ratio of phaeomelanin and eumelanin. The unique melanin concentration ratio value, calculated based on data from a spectral optical sensor and based on thickness data, corresponds to one of the samples in the database of simulated data, the pertinence of such a sample to one of the anthropological types is known. In publication Alaluf S, Atkins D, Barrett K, Blount M, Carter N, Heath A. Ethnic variation in melanin content and composition in photoexposed and photoprotected human skin. Pigment Cell Res. 2002 April; 15(2):112-8. doi: 10.1034/j.1600-0749.2002.1o071. x. PMID: 11936268 it is described how the anthropological type of a human is determined by the ratio of eumelanin and phaeomelanin concentration. Then, according to the selected values, the anthropological type is assigned to the user.

Referring to FIG. 2A, the light from the LEDs is scattered by the user's skin, the scattered radiation falls on photodetectors. The amount of scattered radiation becomes less if it interacts with the melanin of the skin, since the radiation is partially absorbed by melanin. The skin is a kind of filter for radiation depending on the concentration of specific chromophores, the intensity of the radiation transmitted through it changes at wavelengths characteristic of these chromophores. The ratio of scattering peaks from the red, green, and infrared LEDs scattered by the skin carries information about the skin tone and about the anthropological type, depending on the overall melanin concentration, the melanin concentration ratio, and skin thickness.

Skin tone estimation is performed using a machine learning algorithm, in which data from optical scattering signals at operating wavelengths are used as input data, and skin tone is obtained as output data. The machine learning algorithm for determining skin tone is trained on a data set obtained on a computer model of the skin, which allows to determine the scattering spectrum of the skin depending on its parameters, such as melanin concentration and skin thickness. For example, there is a simulated set of spectra (scattering peaks), each spectrum has its own corresponding overall melanin concentration, that is, its own skin tone.

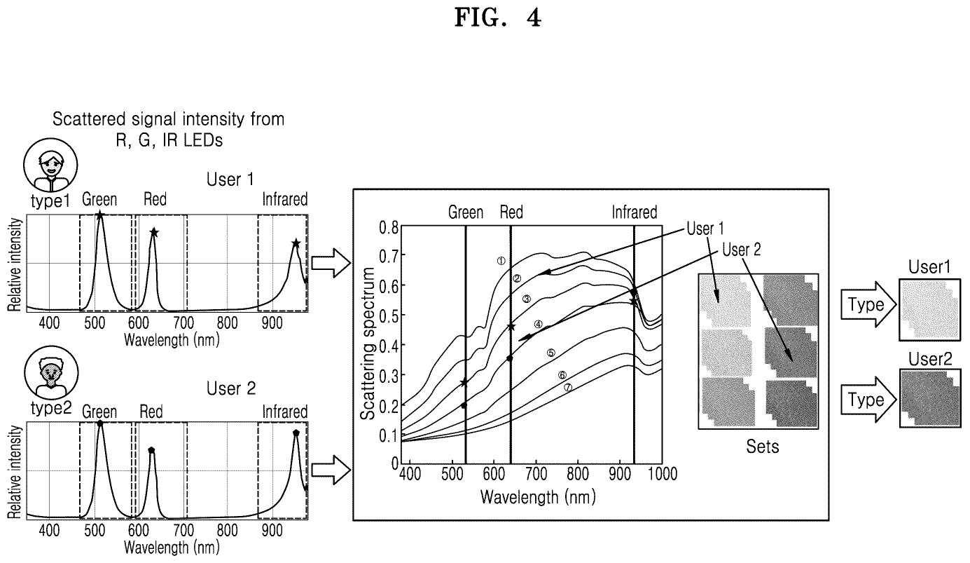

FIG. 4 illustrates a method anthropological type determination for two users of different anthropological types according to an embodiment of the disclosure.

Referring to FIG. 4, the user puts on a smart bracelet, the radiation of R, G, IR of a preset intensity from LEDs turned on sequentially (that is, only one light emitting diode is turned on at a time) penetrates into the user's skin, dissipates, and photodetectors record the intensity values of the scattered radiation for R, G, and IR. It should be noted that radiation of all wavelengths interact with all chromophores of the skin differently, since it is not possible to analytically express concentrations through the intensity of scattered radiation, for this purpose the database of simulated data and the machine learning algorithm are applied, which uses the values of scattering intensities specifically for R, G, and IR LEDs.

The data, that is, three intensity values obtained by photodetectors, are entered into a machine learning algorithm, allowing to determine, which a skin tone the received intensity of each radiation from R, G, and IR, as described above, corresponds to.

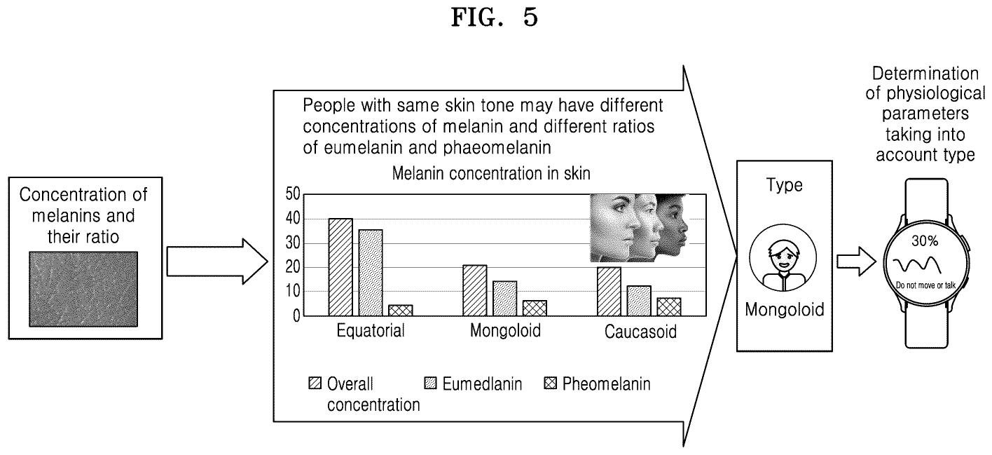

FIG. 5 illustrates a determination of anthropological type of a user depending on an overall melanin concentration and a ratio of eumelanin and phaeomelanin, as presented, for example, in the document Alaluf S, Atkins D, Barrett K, Blount M, Carter N, Heath A according to an embodiment of the disclosure.

Referring to FIG. 5, ethnic variation in melanin content and composition in photoexposed and photoprotected human skin. Pigment Cell Res. 2002 April; 15(2):112-8. doi: 10.1034/j.1600-0749.2002.10071. x. PMID: 11936268. People with the same skin tone may have a different overall melanin concentration and a different ratio between concentrations of eumelanin and phaeomelanin in the skin, by which the anthropological type will be determined. If the chromophore layer is thicker in the skin, then the overall melanin concentration is lower compared to a thinner layer of the same color.

For skin thickness determination it is possible to use any suitable thickness sensor. Information about skin thickness can also be estimated with less accuracy by the user profile, knowing the data of weight, height, user field. Thickness measurement can be carried out using an ultrasonic thickness sensor, known from the prior art. Thickness measurement can be carried out using bioimpedance Birgersson, U., Birgersson, E. and Ollmar, S. “Estimating electrical properties and the thickness of skin with electrical impedance spectroscopy: Mathematical analysis and measurements” Journal of Electrical Bioimpedance, vol. 3, no.1, 2012, pp. 51-60. https://doi.org/10.5617/jeb.400, bioimpedance sensor can also be integrated into a smart bracelet. Bioimpedance is the resistance of biological tissues of the body when an electric current passes through them. Electroimpedance spectroscopy is used for skin thickness determination. When measuring, an electric current is passed through the sample tissue at different frequencies of 1 kHz-1 MHZ, the shape of the spectral impedance curve varies depending on skin thickness, and by analyzing a predefined set of curves for different thicknesses, it is possible to estimate the thickness of the skin under study.

The measurement of skin thickness can also be carried out using a spectral optical sensor, in which a laser is further embedded, as described in the document Wissel T, Bruder R, Schweikard A, Ernst F. Estimating soft tissue thickness from light-tissue interactions—a simulation study. Biomed Opt Express. 2013 Jun. 14;4(7):1176-87. doi: 10.1364/BOE.4.001176. PMID: 23847741; PMCID: PMC3704097. Skin thickness when measured in this way can be determined at a normal incidence of near-infrared laser radiation with a wavelength of 850 nm by analyzing the scattered signal with a standard deviation of 0.1 microns.

To achieve the best performance in spectral measurements, it is preferable to use light emitting diodes operating at wavelengths specially selected for the determination of eumelanin and phaeomelanin. The wavelengths for which the absorption into eumelanin and phaeomelanin differs as much as possible are selected so that it is possible to determine the ratios of eumelanin and phaeomelanin concentration with a minimum error. For example, light emitting diodes emitting at a wavelength of 1450 nm may be used. When using an optical sensor with an added LED emitting at a wavelength of 1450 nm, used to measure the water content in the skin, as well as when using an LED operating at a wavelength of 970 nm, the performance of the user type estimation algorithm increases.

Machine learning algorithms that determine skin tone, skin thickness, general melanin concentration and melanin concentration ratio that determine the user's type can be combined into one machine learning algorithm, the input of which is the intensity values obtained after irradiating the user's skin with LEDs.

Determining the anthropological type of a user can be used to improve the accuracy of estimation of the user's sweating during sports and fitness, to prevent dehydration of the user's organization and to inform the user about the amount of water required for intake, since, for example, according to the conducted research, people belonging to the Mongoloid anthropological type sweat less than people belonging to the Caucasoid antpropological type. Thus, without user type determination, sweat loss forecasts for users belonging to the Mongoloid antpropological type will be overestimated, and for users belonging to the Caucasoid antpropological type will be underestimated. For example, adding the “antpropological type” function to the sweat loss algorithm significantly improves its accuracy.

Thus, the user's anthropological type detection function increases the accuracy of the sweating loss assessment and avoids a situation of risk to the user's health associated with dehydration or hyperhydration caused by inaccurate sweating estimates.

The disclosure can be used to determine the composition of the user's body. The analysis of the bioelectric impedance (BIA) of the human body is based on the properties of the conduction of electric current by the tissues of the body. This smartwatch function is designed to provide the user with information about body composition (amount of fat/muscle/intercellular fluid). Based on the analysis of bioelectric impedance, it is possible to determine the composition of the user's body, particularly the amount of fat, muscle, intercellular fluid. According to the study Jakicic J M, Wing R R, Lang W. Bioelectrical impedance analysis to assess body composition in obese adult women: the effect of ethnicity. Int J Obes Relat Metab Disord. 1998 March; 22(3):243-9. doi: 10.1038/sj.ijo.0800576. PMID: 9539193, body composition, depends on anthropological type of a human. In this paper, an ethnic equation is given to determine the muscle mass of the body for overweight women:

Muscle mass of the body (LBM)=2.68+0.20Ht2/R50+0.19 weight+2.55 ethnicity+0.1157 height,

Ht2/R50—impedance;

Weight—Weight;

*53Ethnicity—coefficient for anthropological type (for equatorial type 1, for Caucasian type 0);

Height—Height.

For example, the muscle mass of the body (LBM) will be calculated inaccurately without taking into account the Anthropological type of human.

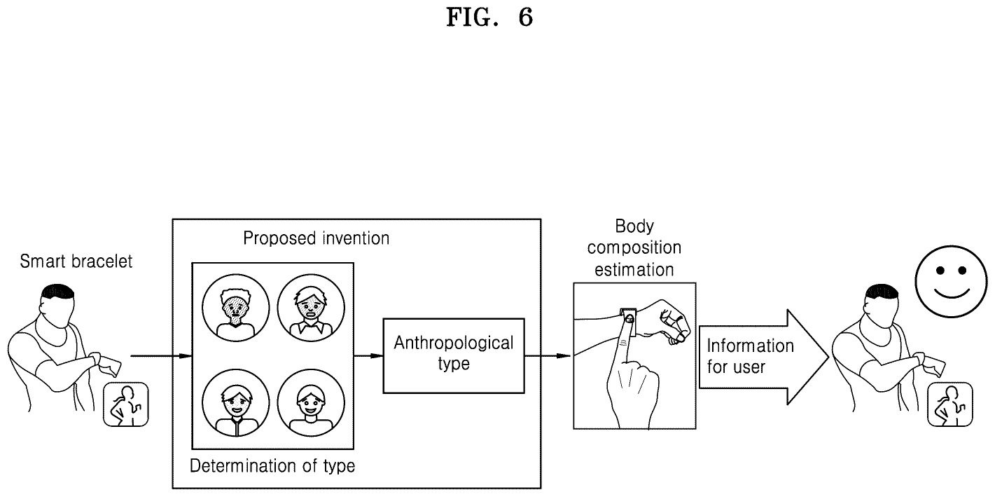

FIG. 6 schematically illustrates an operation of a smart bracelet to measure physiological parameters according to an embodiment of the disclosure.

Referring to FIG. 6, smart bracelets, including fitness bracelets, for determining physiological parameters are very popular among users, for example, smartwatch Samsung Galaxy Watch 4 https://www.samsung.com/ru/watches/galaxy-watch/galaxy-watch4-green-bt-sm-r870nzgacis/they can evaluate the composition of the human body by the bioimpedance method, smartwatch Garmin Vivo Active 4 https://www.garmin.com/en-GB/p/643382/pn/010-02174-02 have the function of estimating sweat loss. Adding the anthropological type determination function to these devices will improve the accuracy of measurements of the ratio of muscle and fat mass and the amount of fluid lost with sweat, respectively. With any user interaction with the smart bracelet, the user's skin tone is determined, skin thickness is measured and the user's anthropological type is determined. When the data is subsequently used to determine, for example, body composition or to estimate fluid loss with sweat during exercise, the obtained results of anthropological type determination are used to refine the user's physiological parameters.

The usual approach, known from the prior art, for taking into account the anthropological type when determining physiological parameters involves transferring to the algorithm for determining the physiological parameter of the user's anthropological type, determined using a direct user questionnaire, which causes user discomfort and may lead to refuse to use the device as a whole. The disclosure allows to determine anthropological type imperceptibly for the user, without causing discomfort.

Another example of the positive effect of adding the “anthropological type determination” function is its use in the algorithm for determining the loss of fluid with sweat during exercise. When developing this algorithm, a number of experiments were conducted to determine the amount of lost fluid in people belonging to different anthropological types, it was found that the inclusion of anthropological type in the set of parameters of the algorithm for determining fluid loss significantly improves the quality of determining fluid loss, since people with different anthropological types lose different amounts of sweat under the same conditions. Without the “anthropological type determination” function, forecasts of body composition, for example, for users of the equatorial anthropological type will be mostly underestimated, and for users of the Caucasian anthropological type-mostly overestimated. Adding the “anthropological type determination” function to the body composition determination algorithm improves its accuracy by about 10%.

The “anthropological type determination” function increases the accuracy of body composition estimation based on BIA and avoids incorrect user actions during weight correction caused by inaccurate results.

Referring to FIG. 6, every time a user initiates a measurement and the skin tone is measured, the machine learning algorithm calculates the user's anthropological type. The machine learning algorithm for measuring physiological parameters uses corrections for calculations, that is, it includes different coefficients for users with different anthropological types.

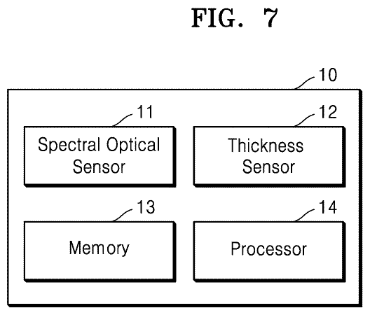

FIG. 7 is a diagram illustrating an apparatus for determining anthropological type of a user according to an embodiment of the disclosure.

Referring to FIG. 7, an apparatus 10 according to an embodiment of the disclosure may include a spectra optical sensor 11, thickness sensor 12, memory 13, and a processor 14.

The spectral optical sensor 11 may emit radiation on the user's skin and obtain scattering intensity values for the radiation on the user's skin. The spectral optical sensor 11 may include photodetectors and light emitting diodes. The light emitting diodes may emit radiation on the user's skin. The photodetectors may obtain scattering intensity values.

The thickness sensor 12 may obtain a thickness value of the user's skin. The thickness sensor may be a bioimpedance sensor, an ultrasonic sensor, or an optical sensor, but not limited thereto.

The processor 14 may calculate an overall melanin concentration value and a melanin concentration ratio value of the user's skin based on the obtained scattering intensity values and the obtained thickness value. The processor 14 may compare the obtained scattering intensity values with values of scattering intensity corresponding to a special skin tone from the database of simulated data, to select a set of skin sample corresponding to the special skin tone from the database of simulated data. The processor 14 may compare the obtained thickness value with data of skin thickness from the database of simulated data, to select skin samples having the closest values with the obtained thickness value from the selected set of skin sample. The processor 14 may compare the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples. The processor 14 may assign an anthropological type to the user in accordance with the selected skin sample.

The spectral optical sensor 11 may obtain scattering intensity values for radiation on the user's skin. The light emitting diodes may emit radiation on the user's skin and the photodetectors may obtain scattering intensity values of the radiation from each of the light emitting diodes on the user's skin.

The thickness sensor 12 may obtain a thickness value of the user's skin. The thickness sensor may be a bioimpedance sensor, an ultrasonic sensor, or an optical sensor, but not limited thereto.

The memory 13 may store one or more instructions and a database of simulated data. The database of simulated data may comprise sets of skin samples. Each set of skin samples may correspond to specific skin tone. The specific skin tone from each set of skin samples may correspond to specific scattering intensity values. Each set of skin samples may comprise skin samples having the same skin tone but differing in skin thickness. Each specific skin sample may correspond to a specific overall melanin concentration value and a specific melanin concentration ratio value. Each specific overall melanin concentration value and specific melanin concentration ratio value of specific skin sample may have corresponding specific anthropological type of a human.

The processor 14 may execute the one or more instructions stored in the memory 13 to determine anthropological type of the user. The processor 14 may calculate an overall melanin concentration value and a melanin concentration ratio value of the user's skin based on the scattering intensity values and the thickness value. The processor 14 may compare the scattering intensity values obtained from the spectral optical sensor 11 and the thickness value obtained from the thickness sensor 12 with the sets of skin samples stored in the database of simulated data, and determine anthropological type of the user based on comparison.

The apparatus 10 for determining anthropological type of a user, including the spectral optical sensor 11 configured to obtain scattering intensity values for radiation on a user's skin, the thickness sensor 12 configured to obtain a thickness value of the user's skin, the memory 13 configured to store one or more instructions, and the processor 14 configured to execute the one or more instructions stored in the memory 13, wherein the memory 13 configured to store a database of simulated data, which comprises sets of skin samples, wherein each set corresponds to specific skin tone, wherein said specific skin tone from each set of skin samples corresponds to specific scattering intensity values, wherein each set of skin samples comprises skin samples, having the same skin tone but differing in skin thickness, wherein each specific skin sample corresponds to a specific overall melanin concentration value and a specific melanin concentration ratio value, and wherein each specific overall melanin concentration value and specific melanin concentration ratio value of specific skin sample have corresponding specific anthropological type of a human, wherein the processor 14 configured to calculate an overall melanin concentration value and a melanin concentration ratio value of the user's skin based on the scattering intensity values and the thickness value, and compare the scattering intensity values obtained from the spectral optical sensor and the thickness value obtained from the thickness sensor with the sets of skin samples stored in the database of simulated data, and determine anthropological type of the user based on comparison.

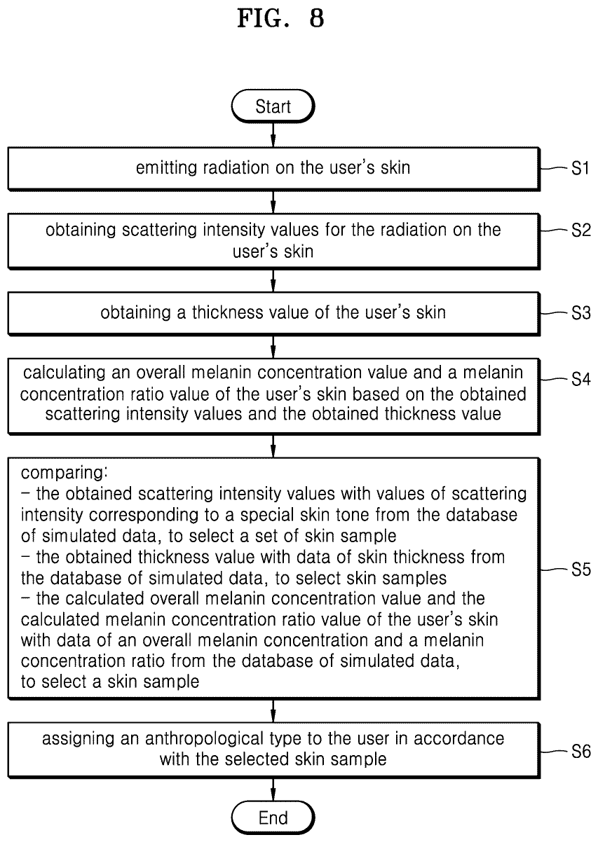

FIG. 8 is a diagram illustrating a method for operating an apparatus for determining anthropological type of a user according to an embodiment of the disclosure.

Referring to FIG. 8, at operation S1, the apparatus may emit radiation on the user's skin. The spectral optical sensor of the apparatus may emit the radiation on the user's skin.

At operation S2, the apparatus may obtain scattering intensity values for the radiation on the user's skin. The spectral optical sensor may obtain the scattering intensity values.

At operation S3, the apparatus may obtain a thickness value of the user's skin. The thickness sensor may obtain the thickness value.

At operation S4, the apparatus may calculate an overall melanin concentration value and a melanin concentration ratio value of the user's skin based on the obtained scattering intensity values and the obtained thickness value. The calculation may be performed by the processor.

At operation S5, the apparatus may compare the obtained scattering intensity values with values of scattering intensity corresponding to a special skin tone from the database of simulated data, to select a set of skin sample corresponding to the special skin tone from the database of simulated data. The apparatus may compare the obtained thickness value with data of skin thickness from the database of simulated data, to select skin samples having the closest values with the obtained thickness value from the selected set of skin sample. The apparatus may compare the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples.

At operation S6, the apparatus may assign an anthropological type to the user in accordance with the selected skin sample.

The method for operating an apparatus for determining anthropological type of a user according to an embodiment of the disclosure may be stored in a computer-readable recording medium by being implemented in the form of program commands that may be performed by various computer means. The computer-readable recording medium may include program instructions, data files, and data structures either alone or in combination. The program commands recorded on the computer-readable recording medium may be those that are especially designed and configured for the disclosure, or may be those that are known and available to computer programmers of ordinary skill in the art. Examples of the computer-readable recording medium may include magnetic media, such as hard disks, floppy disks, and magnetic tapes, optical media, such as compact disc read only memories (CD-ROMs) and digital versatile discs (DVDs), and magneto-optical media, such as floptical disks, and hardware devices, such as read only memories (ROMs), random access memories (RAMs), and flash memories particularly configured to store and execute program commands. Examples of the program commands may include not only machine language code generated by a compiler but also high-level language code that may be executed by a computer by using an interpreter or the like.

The benefits for the user of a smart bracelet are obvious: the performance of physiological parameters evaluation algorithms, such as sweating loss assessment or muscle mass assessment, or the like, improves.

If the smart bracelet is worn by another user, then the smart bracelet, which has the user type determination function, determines that the type of the user wearing the smart bracelet is different from the type of the previous user, and the system of a smart bracelet can, for example, block his work.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An apparatus for determining anthropological type of a user, the apparatus comprising:
a spectral optical sensor configured to emit radiation on a user's skin and obtain scattering intensity values for the radiation on the user's skin;
a thickness sensor configured to obtain a thickness value of the user's skin;
memory storing one or more computer programs; and
one or more processors communicatively coupled to the spectral optical sensor, the thickness sensor, and the memory,
wherein the one or more computer programs include computer-executable instructions that, when executed by the one or more processors, cause the apparatus to:
calculate an overall melanin concentration value and a melanin concentration ratio value of the user's skin based on the obtained scattering intensity values and the obtained thickness value; and
compare:
the obtained scattering intensity values with values of scattering intensity corresponding to a special skin tone from a database of simulated data, to select a set of skin sample corresponding to the special skin tone from the database of simulated data,
the obtained thickness value with data of skin thickness from the database of simulated data, to select skin samples having the closest values with the obtained thickness value from the selected set of skin sample, and
the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples, and assign an anthropological type to the user in accordance with the selected skin sample.

2. The apparatus of claim 1,
wherein the spectral optical sensor comprises light emitting diodes and photodetectors,
wherein the light emitting diodes are configured to emit the radiation on the user's skin, and
wherein the photodetectors are configured to obtain the scattering intensity values of the radiation from each of the light emitting diodes on the user's skin.

3. The apparatus of claim 1, wherein comparison is based on a machine learning algorithm.

4. The apparatus of claim 1, wherein the melanin concentration ratio is a concentration ratio of eumelanin and phaeomelanin.

5. The apparatus of claim 2, wherein the light emitting diodes are configured such that only one of the light emitting diodes is emitted at a time.

6. The apparatus of claim 2, wherein the light emitting diodes comprises a red light emitting diode, a green light emitting diode, and an infrared light emitting diode.

7. The apparatus of claim 1, wherein the thickness sensor is a bioimpedance sensor.

8. The apparatus of claim 1, wherein the thickness sensor is an ultrasonic sensor.

9. The apparatus of claim 1, wherein the thickness sensor includes an optical sensor.

10. A method for operating an apparatus for determining anthropological type of a user, the method comprising:
emitting radiation on a user's skin;
obtaining scattering intensity values for the radiation on the user's skin;
obtaining a thickness value of the user's skin;
calculating an overall melanin concentration value and a melanin concentration ratio value of the user's skin based on the obtained scattering intensity values and the obtained thickness value;
comparing:
the obtained scattering intensity values with values of scattering intensity corresponding to a special skin tone from a database of simulated data, to select a set of skin sample corresponding to the special skin tone from the database of simulated data,
the obtained thickness value with data of skin thickness from the database of simulated data, to select skin samples having the closest values with the obtained thickness value from the selected set of skin sample, and
the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples; and
assigning an anthropological type to the user in accordance with the selected skin sample.

11. The method of claim 10, wherein the melanin concentration ratio includes a ratio of melanin concentrations for eumelanin and phaeomelanin.

12. The method of claim 10, wherein the thickness value is obtained by using data of height, weight, age, and sex of the user.

13. The method of claim 10, wherein the comparing is based on a machine learning algorithm.

14. The method of claim 10, wherein the anthropological type is taken into account while determining user's physiological parameters.

15. One or more non-transitory computer-readable storage media storing computer-executable instructions that, when executed by one or more processors of an apparatus, cause the apparatus to perform operations, the operations comprising:
calculating an overall melanin concentration value and a melanin concentration ratio value of a user's skin based on obtained scattering intensity values and obtained thickness value;
comparing:
scattering intensity values with values of scattering intensity corresponding to a special skin tone from a database of simulated data, to select a set of skin sample corresponding to the special skin tone from the database of simulated data,
the thickness value with data of skin thickness from the database of simulated data, to select skin samples having the closest values with the obtained thickness value from the selected set of skin sample, and
the calculated overall melanin concentration value and the calculated melanin concentration ratio value of the user's skin with data of an overall melanin concentration and a melanin concentration ratio from the database of simulated data, to select a skin sample having the closest value with the calculated overall melanin concentration value and the melanin concentration ratio value from the selected skin samples; and
assigning an anthropological type to a user in accordance with the selected skin sample.

16. The one or more non-transitory computer-readable storage media of claim 15, wherein the melanin concentration ratio includes a ratio of melanin concentrations for eumelanin and phaeomelanin.

17. The one or more non-transitory computer-readable storage media of claim 15, wherein the thickness value is obtained by using data of height, weight, age, and sex of the user.

18. The one or more non-transitory computer-readable storage media of claim 15, wherein comparison is based on a machine learning algorithm.

19. The one or more non-transitory computer-readable storage media of claim 15, wherein the anthropological type is taken into account while determining user's physiological parameters.
